(19) United States
(12) Patent Application Publication
(10) Pub. No.: US 2002/0052649 A1
(43) Pub. Date: May 2, 2002

(54) Graft having region for biological seal formation

(76) Inventor: E. Skott Greenhalgh, Wyndmoor, PA (US)

Correspondence Address: John A. Chionchio, Esquire, Synnestvedt & Lechner LLP, 1101 Market Street, Suite 2600, Philadelphia, PA, 19107-2950, US

(21) Appl. No.: 09/998,961

(22) Filed: Oct. 31, 2001

Related U.S. Application Data
(60) Provisional application No. 60/244,489, filed on Oct. 31, 2000.

(51) Int. Cl. A61F 2/06

(52) U.S. Cl. 623/1.35; 623/1.36; 623/1.47; 623/1.39; 623/1.51

(57) ABSTRACT

A graft compatible with living animal tissue is disclosed. The graft has attachment regions with means for promoting growth of living animal tissue across the attachment regions to form a biological seal between the graft and the tissue. The means for promoting growth include locating pores in the attachment regions sized to favor growth of the tissue, increasing the surface area of the attachment regions by forming filamentary loops extending from the attachment regions, forming the attachment regions from textured filaments, forming the attachment regions from materials which elicit a healing reaction in living animal tissue or coating the attachment regions with a compound such as thrombin or collagen which promotes healing of the tissue.

### RELATED APPLICATION

This application is based on and claims priority of U.S. Provisional Application No. 60/244,489, filed Oct. 31, 2000.

### FIELD OF THE INVENTION

This invention relates to grafts compatible with living animal tissue and having regions which encourage the growth of the tissue across the regions to form a biological seal and attachment between the graft and the tissue.

### BACKGROUND OF THE INVENTION

Grafts are widely used as human implants to treat and correct various disorders, such as a vascular aneurysm, in which the graft replaces a weakened portion of an artery or to repair a hernia, wherein a mesh is used to repair an abnormal opening in the wall of the abdomen through which a portion of the intestine protrudes. Grafts may also find further use in various anastomosis procedures wherein two vessels in the body are joined. Vascular anastomoses include, for example, the arteriovenous shunt, in which an artery is connected to a vein to provide access to the circulatory system for hemodialysis. An ileorectal anastomosis is used to treat ulcerative colitis and involves connecting the ileum to the rectum after a total colectomy. Intestinal anastomoses, such as the Rouxen-Y anastomosis, establish a connection between the intestine and another organ or vessel, such as the stomach or esophagus.

The use of grafts involves attaching and often sealing the graft to the tissue of an organ or vessel. This is presently accomplished by means of stents which support the graft and force it against the tissue;

hooks which are attached to the graft and anchor the graft to the tissue; and sutures wherein the graft is sewn directly to the vessel or tissue. A drawback of these methods of attachment is that they cause trauma to the tissue and damage to the graft which results in leaks occurring at the points of attachment. This is especially problematic in vascular grafts which must withstand repeated pressure pulsations as blood is pumped by the heart. Sutures and hooks tend to cause endoleaks in the connected vessels, which take time to heal and cause major complications if they do not heal and form a proper hemostatic seal.

There is clearly a need for a graft which can be attached to human or other living animal tissue with less trauma and which will readily form a seal between the graft and the tissue which reduces or eliminates leakage at the attachment.

### SUMMARY AND OBJECTS OF THE INVENTION

The invention concerns a graft compatible with living animal tissue. The graft comprises a thin flexible biocompatible substrate having an attachment region engageable with the living animal tissue for attachment of the substrate to the tissue. The attachment region has means for promoting growth of the living animal tissue across the attachment region to sealingly attach the substrate to the tissue.

In one embodiment of the graft, the growth promoting means comprises a plurality of pores extending throughout the attachment region. The pores are sized to promote growth of the living animal tissue within the pores and thereby across the attachment region. Preferably, the substrate comprises a plurality of interlaced filamentary members, the pores being defined by interstices formed between the filamentary members.

In another embodiment of a graft according to the invention, the substrate comprises an elastic, substantially impermeable, non-woven membrane and the growth promoting means again comprises a plurality of pores extending throughout the attachment region, the pores being sized to promote growth of the living animal tissue within the pores and thereby across the attachment region. The pores may be formed by penetrating the substrate or expanding the substrate at the proper rate to produce pores of the desired size.

The growth promoting means may also comprise a textured surface positioned at the attachment region, the textured surface having an increased surface area favoring growth of the living animal tissue across the attachment region.

In another embodiment, the growth promoting means comprises a coating which promotes healing of living animal tissue applied to the attachment region. Such coatings may comprise thrombin, collagen or silicone. This embodiment may be combined with all of the embodiments of the invention to further increase their effectiveness.

In yet another embodiment of the invention, the substrate comprises a plurality of interlaced first filamentary members formed of a first material, and the attachment region comprises a plurality of interlaced second filamentary members formed of a second material different from the first material. The second material has a characteristic eliciting a healing response from living animal tissue. Examples of such material include nylon, polyethylene and polypropylene.

In all of the embodiments, the substrate may be formed in any of a number of practical shapes and preferably comprises an elongated tube having attachment regions positioned at each end.

It is an object of the invention to provide a graft having means for sealingly attaching the graft to living animal tissue while avoiding trauma to the tissue and damage to the graft.

It is another object of the invention to provide a graft having pores sized to promote growth of living animal tissue and form an attachment between the graft and the tissue.

It is yet another object of the invention to provide a graft having attachment regions with increased surface area to promote growth of living animal tissue and form an attachment between the graft and the tissue and form an attachment between the graft and the tissue.

It is again another object of the invention to provide a graft having attachment regions formed by providing a coating which promotes healing of living animal tissue to form an attachment between the tissue and the graft.

It is still another object of the invention to provide a graft having an attachment region comprising a material which elicits a healing response from living animal tissue.

These and other objects and advantages will become apparent upon consideration of the following drawings and detailed description of the preferred embodiments.

### BRIEF DESCRIPTION OF THE DRAWINGS

FIG. 1 shows a partial sectional perspective view of a graft according to the invention;

FIGS. 2-7 each show a partial perspective view of a respective embodiment of a graft according to the invention; and

FIGS. 8 and 9 show each a perspective view of yet other embodiments of the invention.

### DETAILED DESCRIPTION OF THE PREFERRED EMBODIMENTS

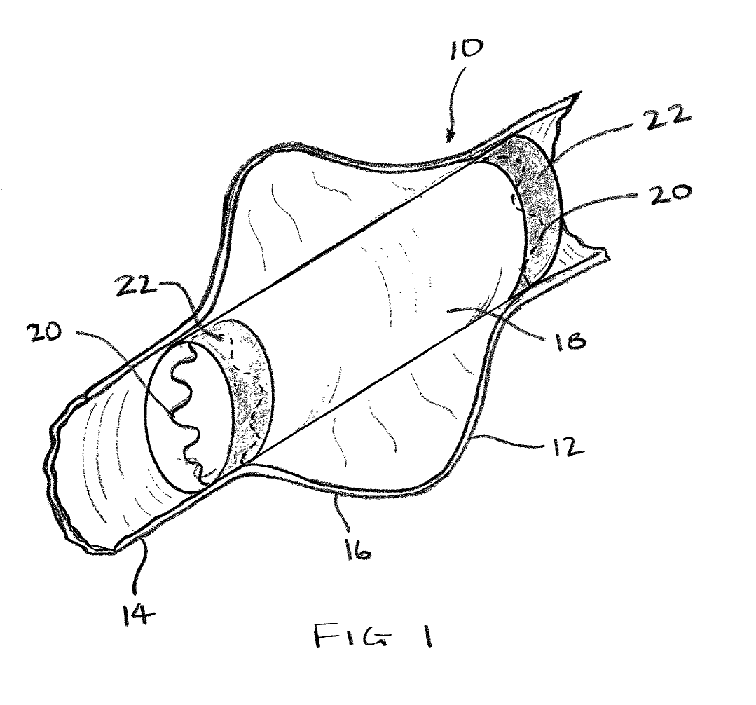

By way of example, FIG. 1 shows a vascular stent graft 10 typically used to treat a vascular aneurysm 12 in an artery 14. (It is understood that the invention is not limited to use with vascular stent grafts but may be applied to any type of graft.) Aneurysm 12 comprises a region of artery 14 wherein the wall 16 is weakened and dilated. Rupture of an aneurysm can be fatal, and treatment consists of replacing the weakened region of the vessel with the stent graft 10. The stent graft may be implanted by means of a catheter or by more invasive surgical techniques.

Stent graft 10 comprises a thin, flexible, biocompatible substrate 18, preferably tubular and having stents 20 located at either end. Stents 20 are spring-like supports which hold the graft open and allow the ends to be sealingly attached to the wall 16 of the artery to either side of aneurysm 12.

At each end of substrate 18 are attachment regions 22 engageable with the living tissue of the artery, the attachment regions having means for promoting growth of the tissue across the attachment region to form a biological attachment and a seal between the graft 10 and the tissue comprising the artery wall 16. The means for promoting growth of the tissue across regions 22 have several embodiments, described in detail below.

### Porous Regions of Higher Permeability

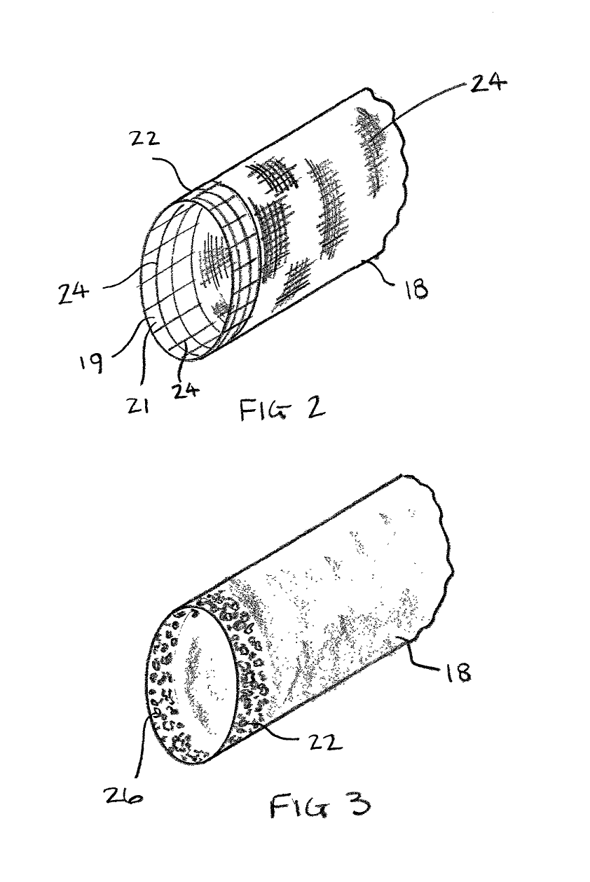

In one embodiment, illustrated in FIGS. 2 and 3, the means for promoting growth of living animal tissue across regions 22 to form the attachment and seal between the graft and the artery comprise relatively narrow bands which have higher permeability than the remaining portion of the graft. The example vascular graft should have a permeability over most of its length no greater than about 300 cc/cm²/min so that it is substantially impermeable and, thus, hemostatic. However, the average pore size associated with this level of permeability is too small and substantially prevents ingrowth of living animal tissue into the graft. Regions 22 are formed which have substantially higher permeability and, consequently, a larger average pore size, so as to accommodate and encourage tissue ingrowth to form a biological seal and attachment. For the vascular graft, a permeability of about 1000 cc/cm²/min for the regions 22 should provide the relatively larger pore sizes which promote substantial tissue ingrowth. Because the substrate 18 between the attachment regions 22 must remain substantially hemostatic, however, the attachment regions 22 of higher permeability are confined to relatively narrow bands preferably between about ⅛ inch and about ¼ inch in length along the substrate. This length should provide adequate attachment and sealing of the graft to the artery without resulting in unacceptable levels of leakage.

As illustrated in FIG. 2, substrate 18 may be formed of interlaced filamentary members 24 and may be woven, knitted or braided. The term “filamentary member” as used herein is a generic term for a continuous strand or strands of fibers, yarns, filaments or material in a form suitable for knitting, weaving, braiding or otherwise intertwining or interlacing to form a fabric. Various forms of filamentary members include monofilaments, filaments twisted together, filaments laid together without twist, as well as other configurations.

For woven grafts, the regions 22 of higher permeability may be formed by changing the density of the weave in these regions, i.e., weaving fewer filamentary members per unit area. For example, regions 22 may be woven with fewer fill yarns per inch, for example, 60 fill yarns per inch, while the remaining length of the graft is woven at 90 fill yarns per inch to maintain hemostatic integrity. Modern looms can readily be programmed to automatically produce tubular sleeves having regions of varying lengths with varying weave density for the manufacture of the graft according to the invention. As shown in FIG. 2, pores 19 providing the increased permeability are defined by interstices 21 formed between the filamentary members 24. Filamentary members comprising polyester are preferred for many grafts due to this material's compatibility with living animal tissue and history of success in human implants. Other materials such as polypropylene, polyethylene and polytetrafluoroethylene are also feasible.

Knitted grafts having regions of varying permeability are also readily manufacturable on modern knitting machines by selectively changing the density of the stitch to include fewer or more loops per unit length as desired to control the permeability and pore size of the graft. Such machines can be programmed to automatically vary the stitch density as a function of graft length by varying the tension under which the yarn is knitted and how much yarn is used per unit length of the graft to produce the regions 22 of higher permeability along with hemostatic regions having relatively high mesh density and consequent low permeability.

As shown in FIG. 3, for substrates 18 comprising elastic, non-woven, continuous membranes formed of polymers such as expanded polytetrafluoroethylene and polyurethane, regions 22 of higher permeability are preferably formed by creating or enlarging pores 26 in narrow bands around the graft. The pores may be formed by mechanically piercing the membrane, etching with chemicals or ablating with a laser.

In a preferred embodiment, a membrane of expanded polytetrafluoroethylene (ePTFE) is used to form the graft substrate 18. As is well known, when polytetrafluoroethylene is heated and stretched quickly to form ePTFE, the heat and the stretching cause the surface to break up and form pores. The pore size can be controlled by the rate at which the polytetrafluoroethylene is stretched. For vascular grafts, the average preferred pore size in the ePTFE membrane over areas other than the attachment regions is about 10 to about 40 microns. This size allows nutrients to pass through the membrane, while ensuring that the graft is hemostatic, but such pores are too small to allow cell ingrowth for sealing and attaching the graft to the vessel. Migration of ePTFE grafts is of particular concern since it is difficult to induce anything to adhere to ePTFE. By including regions 22 of ePTFE comprising pores 26 having relatively greater size than the remaining portion of the graft, areas are created in the graft which will promote the ingrowth of living animal tissue to attach and seal the vessel to the graft via a biological attachment. Pores having an average size between about 100 to about 200 microns in diameter are preferred for the regions 22 and are formed by stretching the regions more rapidly than the adjacent regions of smaller pore size when forming the ePTFE.

### Regions of Increased Surface Area

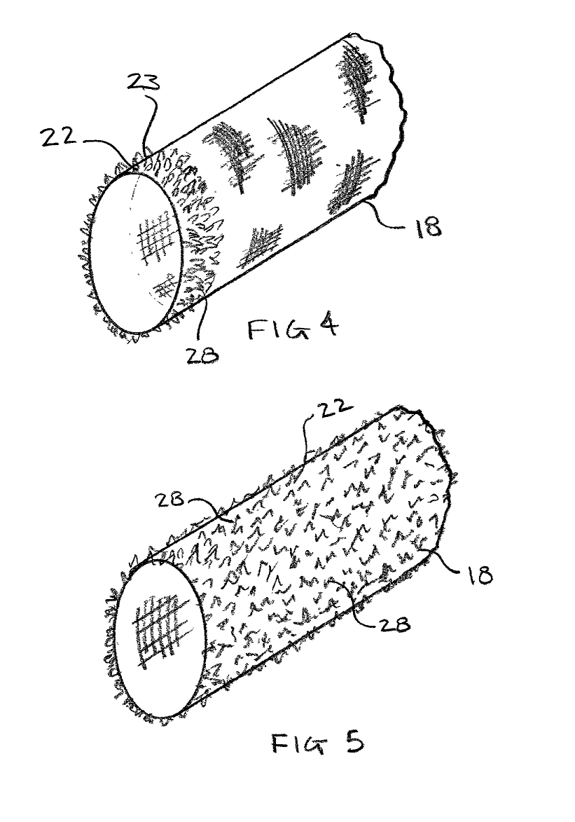

As shown in FIG. 4, a means for promoting growth of living animal tissue across attachment regions 22 to attach and seal the graft to the vessel also includes creating a textured surface 23 having increased surface area. The increased surface area preferably comprises loops 28 which extend outwardly from the substrate 18. The loops may be relatively fine and form a layer of fuzz similar to velour or they may comprise coarser loops, such as found in terry cloth, as well as relatively large loops which are easily visually discernable. The areas of increased surface area comprising regions 22 provide a region which promotes the ingrowth of living animal tissue for attaching and sealing the vessel to the graft.

The loops may be formed in woven or knitted grafts in either the warp or fill directions by well known techniques, such as overfeeding the filamentary members at low tension. Overfeeding pushes more of each filamentary member into the graft and causes the loops to extend outwardly from the substrate. The graft may be formed automatically on a programmable loom or knitting machine by adjusting the tension and overfeeding the filamentary members to produce attachment regions of a predetermined length and then increasing the tension and ceasing overfeeding to produce regions of less bulk and lower surface area. Other methods of forming loops to increase the surface area include adding extra filamentary members in either or both the warp and fill directions to form the regions 22. As shown in FIG. 4a, floats 29, which are warp or fill filamentary members which are not interwoven over the regions 22 but ride on the surface of the fabric, may also be used to selectively create textured surface 23 which encourages tissue ingrowth. As shown in FIG. 4b, the textured surface 23 having increased surface area may also be formed over region 22 by weaving or knitting textured filamentary members 31 under varying tension, using low tension where the fabric is to be bulkier and have a higher surface area and high tension to stretch the yarns and remove the bulk and reduce the surface area available for attachment to the living animal tissue.

The density of the loops is controlled by limiting the number of filamentary members used to form the loops. For example, for a vascular graft having 300 warp yarns, 60 of the yarns may be used to form the loops. The 60 loop yarns are preferably evenly spaced circumferentially around the graft and will provide adequate surface area for tissue ingrowth without adding too much bulk to the graft. (Bulky grafts cannot be implanted by means of a catheter, and too much bulk is to be avoided for catheter delivered grafts.) The density of the loops may be increased or decreased by increasing or decreasing the percentage of loop forming yarns in the graft or by adding more or fewer overfed yarns to form the loop yarns. If fill yarns are to form the regions of increased surface area, the loop density is similarly controlled by the number of fill yarns overfed under low tension or added to the fabric comprising the substrate.

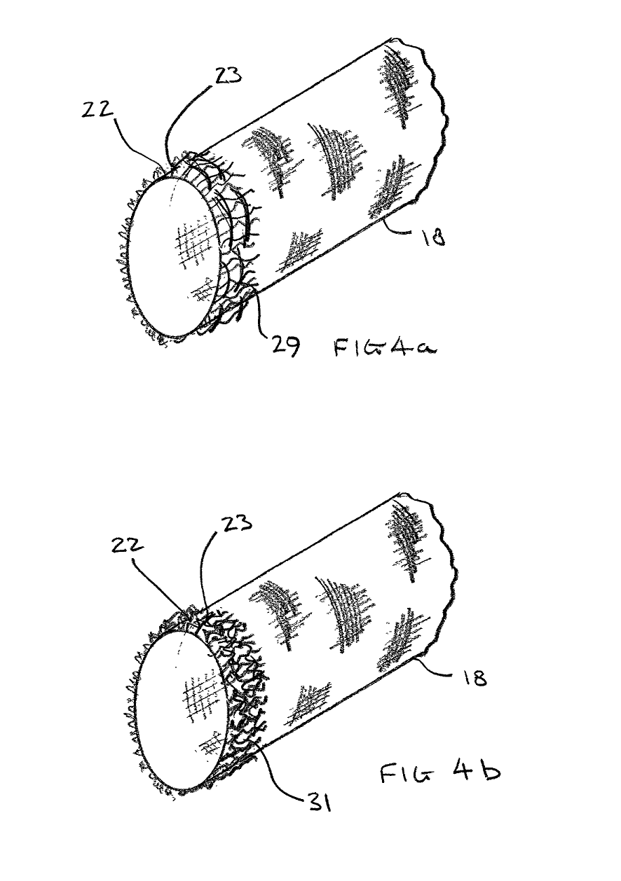

One advantage with using increased surface area to form the regions to promote living animal tissue ingrowth is that the hemostatic properties of the graft are maintained throughout the entire length of the graft, i.e., there are no regions of high permeability which may leak initially after the graft is implanted and require time to heal. This characteristic allows the regions 22 to be extended to cover the length of the graft so that the entire graft becomes a substrate for the ingrowth of tissue as shown in FIG. 5. Such a graft would be useful in the treatment of aneurysms, which sometimes shrink after a graft has been implanted to relieve the pressure. As the aneurysm shrinks, the endothelial cells lining the artery wall contact the graft and will be encouraged to grow into it due to the presence of the loops 28 extending from the substrate 18.

### Coating Graft with a Bioactive Substance

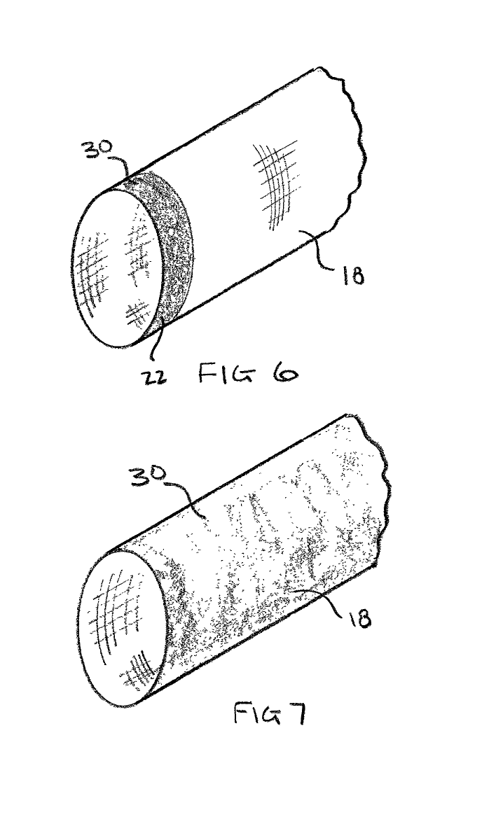

Yet another means of promoting living animal tissue ingrowth with a graft is by coating the graft with bioactive substances which cause aggressive healing or blood clotting response when they are in contact with living animal tissue or blood. Examples of such substances are thrombin, collagen and silicone. As shown in FIG. 6, the bioactive substance may be applied as a coating 30 to selected regions 22 of the substrate 18 or over the entire surface of the substrate (see FIG. 7) by various methods such as painting, dipping and spraying. The filamentary members themselves could also be coated or impregnated with the substance prior to weaving, knitting or braiding.

While the bioactive substance alone may be used to effect the attachment and seal of the graft to the tissue, it is preferred to use it in combination with the above described embodiments by coating the regions of higher permeability or textured surfaces having increased surface area with the bioactive substance to promote rapid initial tissue ingrowth at these locations. This should shorten the time required for the graft to join to the tissue and reduce the incidence of leakage for vascular grafts.

### Include Materials Which Encourage Healing and Clotting

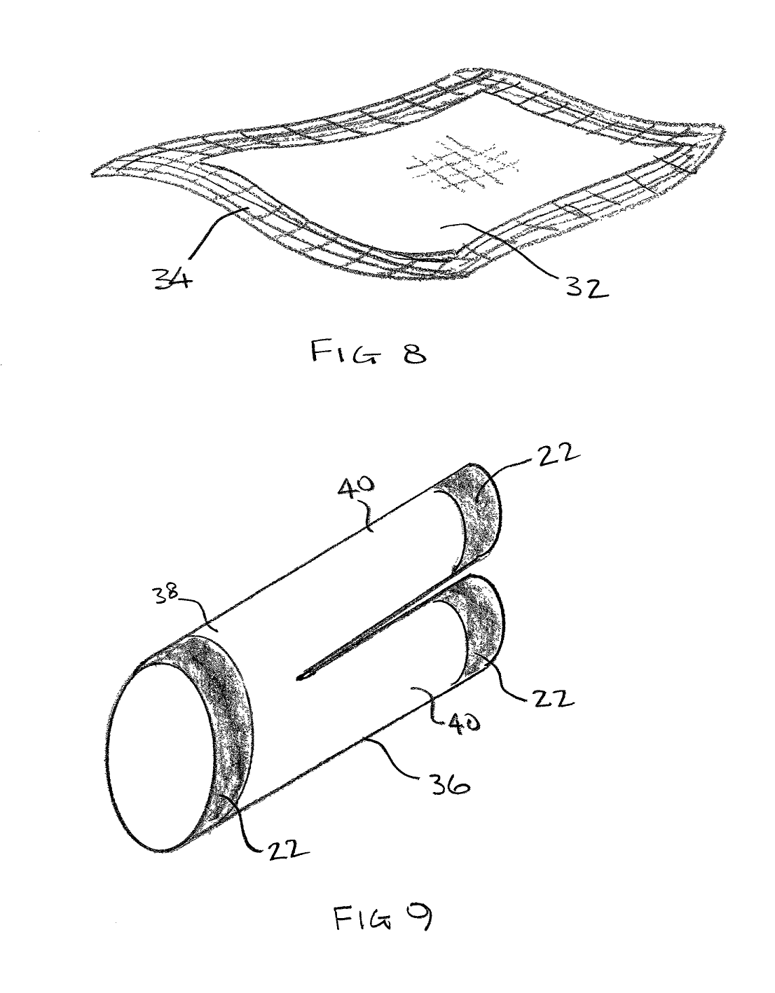

Materials such as nylon, polypropylene and polyethylene elicit a natural healing response when in contact with living animal tissue and, thus, provide yet another means for promoting tissue growth in attachment regions of a graft. The tissue forms around the material to encapsulate and isolate it from the body. By selectively positioning such material in the graft, tissue will be encouraged to attach and seal itself to the graft in these regions. To further promote tissue ingrowth, the material may be woven or knitted having higher permeability or with loops providing greater surface area for the attachment of the cells. FIG. 8 shows an example of a flat mesh graft 32 used to repair hernias. The graft 32 may comprise PTFE filaments interlaced with nylon in perimeteral regions 34 which will elicit the healing reaction of the tissue and promote more rapid ingrowth of tissue into the graft 32 at the perimeter 34.

FIG. 9 shows yet another example of a graft according to the invention, the graft being a bifurcated sleeve 36 for the treatment of abdominal aortic aneurysms. Such aneurysms occur in the abdominal aorta between the renal and iliac arteries and require a bifurcate graft to accommodate the branching of the aorta into the iliac arteries. Regions 22 which form the attachment and seal between the graft and the arteries are positioned at the end of the main tube 38 and each of the branch tubes 40. In the example of FIG. 9, no particular embodiment of the regions 22 is specified. As with any of the grafts described, the regions could comprise any of the aforementioned embodiments such as regions of higher permeability, increased surface area, bioactive coatings or materials which encourage healing. The various regions 22 need not all be the same type of region, the graft 36 could employ a combination of the aforementioned embodiments in the regions 22 as necessary to effect the attachment.

Grafts having regions which promote the ingrowth of living animal tissue and the formation of biological attachments and seals promise to improve the treatment of various disorders by reducing the trauma to the tissue occasioned by the implanting of the graft, reducing the amount of endoleakage at the graft and shortening the healing time required.

What is claimed is:

1. A graft compatible with living animal tissue, said graft comprising a thin flexible substrate having an attachment region biocompatible with said tissue, said attachment region being engageable with said living animal tissue for attachment of said substrate thereto, said attachment region having means for promoting growth of said living animal tissue across said attachment region to sealingly attach said substrate to said tissue.

2. A graft according to claim 1, wherein said growth promoting means comprises a plurality of pores extending throughout said attachment region, said pores being sized to promote growth of said living animal tissue within said pores and thereby across said attachment region.

3. A graft according to claim 2, wherein said substrate comprises a plurality of interlaced filamentary members, said pores being defined by interstices formed between said filamentary members.

4. A graft according to claim 3, wherein said substrate comprises an elongated tube, one of said attachment regions being positioned at each end of said tube.

5. A graft according to claim 4, wherein said tube is a bifurcated tube.

6. A graft according to claim 4, wherein said filamentary members are interlaced by weaving, said filamentary members comprising said attachment regions being woven with fewer filamentary members per unit area than said filamentary members comprising a portion of said tube between said attachment regions, thereby providing relatively larger interstices over said attachment regions and forming said pores adapted to promote growth of said living animal tissue across said attachment regions, said portion of said tube between said attachment regions having interstices sized relatively smaller, thereby making said portion between said attachment regions substantially impermeable to fluids allowing said tube to act as a fluid conduit.

7. A graft according to claim 6, wherein said pores extending throughout said attachment regions are sized to provide a permeability of about 1000 cc/cm2/min for promoting growth of said living animal tissue across said attachment regions, said portion of said tube between said attachment regions having a permeability of about 300 cc/cm2/min and being substantially fluid impermeable.

8. A graft according to claim 6, wherein said filamentary members comprising said attachment region have a coating which promotes healing of living animal tissue.

9. A graft according to claim 8, wherein said coating is selected from the group consisting of thrombin, collagen and silicone.

10. A graft according to claim 1, wherein said substrate comprises an elastic, non-woven membrane and said growth promoting means comprises a plurality of pores extending throughout said attachment region, said pores being sized to promote growth of said living animal tissue within said pores and thereby across said attachment region.

11. A graft according to claim 10, wherein said pores are formed by piercing said membrane throughout said attachment region.

12. A graft according to claim 11, wherein said membrane comprises an elongated tube, one of said attachment regions being positioned at each end of said tube.

13. A graft according to claim 12, wherein said pores have an average size between about 100 microns and about 200 microns in diameter.

14. A graft according to claim 12, wherein said membrane comprising said attachment regions has a coating which promotes healing of living animal tissue.

15. A graft according to claim 14, wherein said coating is selected from the group consisting of thrombin, collagen and silicone.

16. A graft according to claim 2, wherein said substrate comprises a thin flexible membrane of expanded polytetrafluoroethylene, said membrane being expanded in said attachment region at an expansion rate adapted to form said pores sized to promote growth of said living animal tissue across said attachment region.

17. A graft according to claim 16, wherein said membrane comprises an elongated tube, one of said attachment regions being positioned at each end of said tube, said membrane between said attachment regions being formed by expanding said polytetrafluoroethylene at a second expansion rate relatively lower than said first named expansion rate thereby yielding a substantially impermeable tube between said attachment regions.

18. A graft according to claim 17, wherein said membrane comprising said attachment regions has a coating which promotes healing of living animal tissue.

19. A graft according to claim 18, wherein said coating is selected from the group consisting of thrombin, collagen and silicone.

20. A graft according to claim 17, wherein said pores in said attachment region have an average size between about 100 microns to about 200 microns in diameter.

21. A graft according to claim 1, wherein said growth promoting means comprises a textured surface positioned at said attachment region, said textured surface having an increased surface area favoring growth of said living animal tissue across said attachment region.

22. A graft according to claim 21, wherein said textured surface comprises a plurality of loops extending outwardly from said substrate, said loops providing said increased surface area favoring growth of said living animal tissue.

23. A graft according to claim 22, wherein said substrate comprises a plurality of interlaced filamentary members, said filamentary members being overfed during interlacing at least in said attachment region to form said loops extending outwardly to form said textured surface.

24. A graft according to claim 22, wherein said substrate comprises a plurality of filamentary members interlaced by weaving and said loops comprise floats positioned at least in said attachment region and extending outwardly to form said textured surface.

25. A graft according to claim 21, wherein said substrate comprises a plurality of interlaced filamentary members, said filamentary members being textured filamentary members at least in said attachment region, said textured filamentary members having increased bulk providing said increased surface area favoring growth of said living animal tissue.

26. A graft according to claim 21, wherein said textured surface comprising said attachment region has a coating which promotes healing of living animal tissue.

27. A graft according to claim 26, wherein said coating is selected from the group consisting of thrombin, collagen and silicone.

28. A graft according to claim 1, wherein said attachment region comprises a surface having a coating which promotes healing of living animal tissue.

29. A graft according to claim 28, wherein said coating is selected from the group consisting of thrombin, collagen and silicone.

30. A graft according to claim 1, wherein said substrate comprises a plurality of interlaced first filamentary members formed of a first material, said attachment region comprising a plurality of interlaced second filamentary members formed of a second material different from said first material, said second material having a characteristic eliciting a healing response from living animal tissue.

31. A graft according to claim 30, wherein said second material is selected from among the group consisting of nylon, polypropylene and polyethylene.
